(19) United States
(12) Patent Application Publication
(10) Pub. No.: US 2004/0136937 A1
(43) Pub. Date: Jul. 15, 2004

(54) Compositions comprising a tensioning polymer and an ionic amphiphilic polymer

(75) Inventor: Guillaume Cassin, Villebon Sur Yvette (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL, Paris (FR)

(21) Appl. No.: 10/706,711

(22) Filed: Nov. 12, 2003

Related U.S. Application Data
(60) Provisional application No. 60/428,743, filed on Nov. 25, 2002.

(30) Foreign Application Priority Data
Nov. 12, 2002 (FR) 0214116

(51) Int. Cl. A61K 7/06; A61K 7/11

(52) U.S. Cl. 424/70.12; 424/70.16; 424/70.17

(57) ABSTRACT

The invention relates to a composition that is suitable for topical application to the skin, comprising an aqueous phase, a fatty phase, a dispersion of tensioning polymer particles that exhibit a retraction of isolated stratum corneum of 1.5% when tested at 30° C., at a relative humidity of 40%, and at a concentration of 7% in water, and at least one ionic amphiphilic polymer.

### CROSS-REFERENCE TO RELATED APPLICATIONS

The present application claims the benefit of U.S. Provisional Application No. 60/428,743, the disclosure of which is incorporated by reference herein.

### BACKGROUND OF THE INVENTION

The present invention relates to a composition that is suitable for topical application to the skin, containing an aqueous phase, a fatty phase, a dispersion of tensioning polymer particles and at least one ionic amphiphilic polymer. The entire content of French foreign application no. 0214116, filed Nov. 12, 2002, is hereby incorporated by reference.

In the course of the aging process, various signs appear on the skin that are very characteristic of this aging, reflected especially by a change in the structure and functions of the skin. The main clinical signs of aging of the skin are especially the appearance of fine lines and deep wrinkles, which increase with age. Disruption of the “grain” of the skin is observed in particular, i.e. the microrelief is less uniform and has an anisotropic nature.

It is known practice to treat these signs of aging using cosmetic or dermatological compositions containing active agents capable of combating aging, such as α-hydroxy acids, β-hydroxy acids and retinoids. These active agents act on wrinkles by removing the dead cells from the skin and by accelerating the process of cell renewal. However, these active agents have the drawback of being effective in treating wrinkles only after they have been applied for a certain time. It is increasingly sought to obtain an immediate effect for the active agents used.

To this end, in recent years agents with a tensioning effect have been proposed, which, immediately after application, smooth out the wrinkles and fine lines and contribute towards attenuating fatigue marks. These compounds act by forming a film that causes retraction of the stratum corneum, the superficial outermost layer of the epidermis. Examples of such tensioning agents are especially aqueous dispersions of synthetic polymers (WO 98/29092), in particular of “starburst” polymers (EP-1 043 345), of grafted silicone polymer (EP-1 038 519) or of polymer comprising units with an LCST.

However, these prior-art tensioning agents are rarely formulated in the presence of oil, in particular in O/W or W/O emulsions, but more often are in the form of sera, i.e. gelled aqueous compositions. The reason for this is that it is generally observed that the efficacy of these compounds is greatly reduced in the presence of oil.

However, the presence of an oily phase in a cosmetic composition is often desirable, whether to give the composition an emollient nature or to convey various liposoluble topical active agents, such as vitamins A and E, especially, which are particularly useful in anti-wrinkle compositions.

There is thus still a need for a composition with a tensioning effect, containing an aqueous phase, an oily phase and a dispersion of particles of tensioning polymer, which is stable over time, which is for commercial use, and which offers skin-tensioning properties that are sufficient to visibly smooth out wrinkles and fine lines immediately on application.

### SUMMARY OF THE INVENTION

Now, the Applicant has discovered, surprisingly, that the reduction of the tensioning power of latices when they are formulated in emulsion form is due to the presence of surfactants in these emulsions, and that by replacing these surfactants, totally or partially, with one or more ionic amphiphilic polymer(s), it is possible to obtain an emulsion that has a better tensioning effect than those of the prior art, without loss of stability of the emulsion.

One aspect of the invention is thus a composition that is suitable for topical application to the skin, containing an aqueous phase, a fatty phase, a dispersion of tensioning polymer particles, and at least one ionic amphiphilic polymer. Second aspect of the invention is a composition that is suitable for topical application to the skin, containing an aqueous phase, fatty phase, a dispersion of tensioning polymer particles, 1% or less of surfactant, and at least one ionic amphiphilic polymer.

Third aspect of the invention is a method for treating wrinkled skin, comprising applying to the skin the composition described above.

Fourth aspect of the invention is a method for restoring skin tautness, comprising applying to the skin the composition described above.

### DETAILED DESCRIPTION OF THE INVENTION

As used herein, a “tensioning polymer” is one that produces, at a concentration of 7% by weight in water, a retraction of isolated stratum corneum of more than 1.5% at 30° C. and under a relative humidity of 40%, as measured in accordance with the protocol set forth in Example 5.

The dispersion of tensioning polymer particles used according to the invention, also known as a “latex”, preferably consists of a synthetic polymer and may consist of polyurethane polymers and copolymers, in particular polyester-polyurethane copolymers or polyether-polyurethane copolymers; acrylic polymers and copolymers; or sulphonated isophthalic acid polymers.

These polymers may be in the form of polycondensates, hybrid polymers and interpenetrated polymer networks (IPNs).

For the purposes of the present invention, the expression “interpenetrated polymer network” means a blend of two interlaced polymers, obtained by simultaneous polymerization and/or crosslinking of two types of monomer, the blend obtained having a single glass transition temperature.

Examples of IPNs that are suitable for use in the present invention, and also the process for preparing them, are described in U.S. Pat. Nos. 6,139,322 and 6,465,001, for example.

Preferably, the IPN according to the invention comprises at least one polyacrylic polymer and more preferably also comprises at least one polyurethane or one copolymer of vinylidene fluoride and of hexafluoropropylene.

According to one preferred form, the IPN according to the invention comprises a polyurethane polymer and a polyacrylic polymer. Such IPNs are especially those of the Hybridur series that are commercially available from the company Air Products.

An IPN that is particularly preferred is in the form of an aqueous dispersion of particles with a weight-average size of between 90 and 110 nm and a number-average size of about 80 nm. This IPN preferably has a glass transition temperature, Tg, ranging from about −60° C. to +100° C. An IPN of this type is sold especially by the company Air Products under the trade name Hybridur X-01602. Another IPN that is suitable for use in the present invention is referenced Hybridur X18693-21.

Other IPNs that are suitable for use in the present invention comprise IPNs consisting of a blend of a polyurethane with a copolymer of vinylidene fluoride and of hexafluoropropylene. These IPNs may be prepared especially as described in U.S. Pat. No. 5,349,003. As a variant, they are commercially available in the form of a colloidal dispersion in water, in a ratio of the fluorinated copolymer to the acrylic polymer of between 70:30 and 75:25, under the trade names Kynar RC-10, 147 and Kynar RC-10, 151 from the company Atofina.

Polymers in the form of polycondensates have been described especially in patent application (WO 98/29092). They comprise in particular those sold under the trade names Avalure UR410 and Avalure UR460 by the company Noveon, and under the trade names Neorez R974, Neorez R981 and Neorez R970 by the company Avecia, and also the copolymer Neocryl XK-90 sold by the company Avecia.

The dispersion of polymer particles used according to the invention may as a variant consist of a grafted silicone polymer, as defined in patent application EP-1 038 519, and more particularly a polymer comprising a main silicone chain (or polysiloxane (≡Si—O—)ₙ) onto which is grafted, within the said chain and also optionally on at least one of its ends, at least one silicone-free organic group.

A preferred example of a grafted silicone polymer is polysilicone-8 (CTFA name), which is a polydimethylsiloxane onto which are grafted, via a linking chain of thiopropylene type, mixed polymer units of the poly(meth)acrylic acid type and of the polyalkyl (meth)acrylate type. A polymer of this type is available especially under the trade name VS 80 (at 10% in water) or LO 21 (in pulverulent form) from the company 3M. It is a copolymer of polydimethylsiloxane containing propylthio groups, of methyl acrylate, of methyl methacrylate and of methacrylic acid.

According to yet another possibility, the latex used according to the invention may consist of a polymer of “starburst” structure represented by formula (I) below:

A-[(M1)ₚ₁-(M2)ₚ₂ . . . (Mi)ₚⱼ]n (I)

in which:

A represents a multifunctional centre, of functionality “n”, n being an integer greater than 2,

[(M1)ₚ₁-(M2)ₚ₂ . . . (Mi)ₚⱼ] represents a polymer chain, also referred to as a “branch”, consisting of polymerized monomers Mi, which may be identical or different, having a polymerization index pj, each branch being identical or different, and being covalently grafted onto the said centre A,

i being greater than or equal to 1, and pj being greater than or equal to 2; the said polymer comprising one or more monomers Mi whose corresponding homopolymer has a Tg of greater than or equal to about 10° C., preferably greater than or equal to 15° C. and better still greater than or equal to 20° C.;

this or these monomer(s) Mi being present, in the final polymer, in a minimum amount of about 45% by weight, preferably in an amount of between 55% and 99% by weight and better still in an amount of 75-90% by weight, relative to the total weight of monomers.

These polymers, and the process for preparing them, are described in patent application EP-1 043 345.

The latices or dispersions of tensioning polymer particles that may be used in the present invention are not limited to the abovementioned types, but may consist of any type of polymer having a sufficient tensioning effect, characterized by the stratum corneum retraction value defined above. This retraction value is measured using an extensometer, according to the method described in Example 5 below.

The amount of tensioning polymer present in the composition may vary within a wide range as a function of the desired effect. By way of example, the tensioning polymer may represent from 0.1% to 50% by weight and preferably from 1% to 20% by weight, relative to the total weight of the composition.

As indicated above, the composition according to the invention also contains an ionic amphiphilic polymer.

This expression means cationic, anionic or amphoteric polymers comprising both a hydrophilic portion and a hydrophobic portion and having the property of forming a film that separates two liquids of different polarity, thus making it possible to stabilize liquid-liquid dispersions of direct, inverse or multiple type. More specifically, the amphiphilic polymers according to the invention have the property of reducing the water/oil interface tension to 10 mN/m, irrespective of the oil. They may be water-soluble or water-dispersible. The term “water-soluble” means that they can be dispersed in water in the form of a molecular solution. The term “water-dispersible” means that they can be dispersed in water in particulate form.

The amphiphilic polymers in accordance with the invention generally have a number-average molecular weight ranging from 1 000 to 20 000 000 g/mol, preferably ranging from 20 000 to 8 000 000 g/mol and even more preferably from 100 000 to 700 000 g/mol.

The ionic amphiphilic polymers used according to the invention preferably do not form cohesive films on drying. The expression “formation of a cohesive film” means that, after deposition onto a glass plate and evaporation of the solvent, a macroscopic film is formed, which can be peeled from the glass plate and manipulated.

Examples of ionic amphiphilic polymers that are suitable for use in the present invention will now be described in greater detail.

Water-soluble ionic amphiphilic polymers

A-1-Acrylic copolymers

The acrylic copolymers that may be used as ionic amphiphilic polymers according to the invention are compounds obtained by copolymerization of a monomer (a) chosen from α,β-ethylenically unsaturated carboxylic acids with a monomer (b) derived from the reaction of an ethylenically unsaturated carboxylic acid monomer with an optionally alkoxylated, in particular polyethoxylated, aliphatic fatty alcohol the carbon-based chain of which contains at least 6 carbon atoms.

According to a first variant, these acrylic copolymers may be chosen from optionally crosslinked and/or neutralized copolymers, consisting of a major fraction of C₃-C₆ monoolefinically unsaturated carboxylic acid monomer or the anhydride thereof and of a minor fraction of a fatty-chain acrylic acid ester monomer. These copolymers are prepared by polymerizing a predominant amount of monoolefinically unsaturated carboxylic acid monomer or anhydride thereof with a smaller amount of fatty-chain acrylic ester monomer. The term “fatty-chain” means a linear or branched alkyl radical containing from 8 to 30 carbon atoms.

The amount of carboxylic acid monomer or anhydride thereof preferably ranges from 80% to 98% by weight and more preferably from 90% to 98% by weight, while the acrylic ester is present in amounts ranging from 2% to 20% by weight and more preferably from 1% to 10% by weight, the percentages being calculated relative to the total weight of the two monomers.

The preferred carboxylic monomers are chosen from those corresponding to formula (II) below:

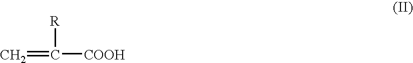

in which R denotes hydrogen, a halogen, a hydroxyl group, a lactone group, a lactam group, a cyanogen group (—C═N), a monovalent alkyl group, an aryl group, an alkylaryl group, an aralkyl group or a cycloaliphatic group.

The fatty-chain acrylic ester monomers are preferably chosen from those corresponding to formula (III) below:

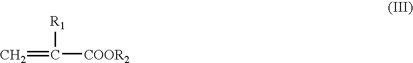

in which R₁ is chosen from the group formed by hydrogen, a methyl radical and an ethyl radical, and R₂ is a C₈-C₃₀ alkyl group.

The polymers formed from a mixture of monomers comprising acrylic acid and an ester of formula (III) in which R1 denotes H or CH₃, R₂ denoting an alkyl radical containing from 12 to 22 carbon atoms, and a crosslinking agent, are more preferably used.

The acrylic copolymers that are preferred for use in the present invention consist of 95% to 60% by weight of acrylic acid, 4% to 40% by weight of C₁₀-C₃₀ alkyl acrylate and 0 to 6% by weight of crosslinking polymerizable monomer, or 98% to 96% by weight of acrylic acid, 1% to 4% by weight of C₁₀-C₃₀ alkyl acrylate, and 0.1% to 0.6% by weight of crosslinking polymerizable monomer.

The crosslinking agent may be used in an amount ranging from 0.1% to 4% and preferably from 0.2% to 1% by weight relative to the total weight of carboxylic monomers and of acrylic ester monomers. The crosslinking agent may be chosen especially from polymerizable monomers containing a polymerizable CH₂═C— group and at least one other polymerizable group, the unsaturated bonds of which are not conjugated relative to each other. A crosslinking polymerizable monomer that is preferred for use in the present invention is chosen from: polyallylsucrose and polyallylpentaerythritol.

These copolymers are described in document EP-A-0 268 164 and are obtained according to the preparation methods described in the document.

The polymers in accordance with the invention are preferably partially or totally neutralized with a mineral base (for example sodium hydroxide, potassium hydroxide or aqueous ammonia) or an organic base such as monoethanolamine, diethanolamine, triethanolamine, aminomethylpropanediol, N-methylglucamine, and basic amino acids, for instance arginine and lysine, and mixtures thereof.

Preferred examples of acrylic copolymers that may be used in the present invention are the acrylate/C₁₀-C₃₀-alkylacrylate copolymers (CTFA name) sold under the names Pemulen TR1, Pemulen TR2 and Carbopol 1382 by the company Goodrich, or mixtures thereof.

According to a second variant, the acrylic copolymer that may be used as ionic amphiphilic polymer according to the present invention may be obtained by copolymerization of a monomer (a) chosen from α,β-ethylenically unsaturated carboxylic acids with a monomer (b) derived from the reaction of itaconic acid with an alkoxylated, in particular polyethoxylated, aliphatic fatty alcohol, comprising from 1 to 50 alkylene oxide units, the carbon-based chain of which contains at least 6 carbon atoms.

Such a copolymer may be a copolymer of acrylic acid and of oxyethylenated (20 EO) monostearyl itaconate or a copolymer of acrylic acid and of oxyethylenated (20 EO) monocetyl itaconate. Such copolymers are sold under the names Structure 2001 and Structure 3001 by the company National Starch.

A-2-Hydrophobic AMPS derivatives

The ionic amphiphilic polymers according to the invention may be, inter alia, amphiphilic polymers comprising at least one ethylenically unsaturated monomer containing a sulphonic group, in freeform or partially or totally neutralized form and comprising at least one hydrophobic portion. The hydrophobic portion present in these polymers preferably contains from 6 to 50 carbon atoms, preferably from 6 to 22 carbon atoms, more preferably from 6 to 18 carbon atoms and even more preferably from 12 to 18 carbon atoms.

These polymers may be non-crosslinked polymers based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C₆-C₁₈ linear alkyl radicals (for example n-hexyl, n-octyl, n-decyl, n-hexadecyl, n-dodecyl or oleyl), branched alkyl radicals (for example isostearic) or cyclic alkyl radicals (for example cyclododecane or adamantane), C₆-C₁₈ fluoro or alkylfluoro radicals (for example the group of formula —(CH₂)₂—(CF₂)₉—CF₃), a cholesteryl radical or radicals derived from cholesterol (for example cholesteryl hexanoate), aromatic polycyclic groups, for instance naphthalene or pyrene, and silicone or alkylsilicone or alkylfluorosilicone radicals. Among these radicals, linear and branched alkyl radicals are more particularly preferred.

These polymers are water-soluble or water-dispersible in neutralized form. Their viscosity at a concentration of 1% in water, at a shear rate of 1 s⁻¹, at a pH of between 5 and 8, at 25° C., is less than 5 000 mPa.s. Polymers of the invention may be chosen especially from the non-crosslinked amphiphilic polymers described in documents EP 1 138 703 and EP 1 069 142.

The amphiphilic polymers according to the invention may also be crosslinked. The crosslinking agents may be chosen from the polyolefinically unsaturated compounds commonly used for crosslinking polymers obtained by free-radical polymerization. According to one preferred embodiment of the invention, the crosslinking agent is chosen from methylenebisacrylamide, allyl methacrylate or trimethylolpropane triacrylate (TMPTA). The degree of crosslinking generally ranges from 0.01 mol % to 10 mol % and more preferably from 0.2 mol % to 2 mol % relative to the polymer.

The preferred polymers of the invention are chosen from crosslinked or non-crosslinked amphiphilic polymers of 2acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, more preferably from 6 to 18 carbon atoms and even more preferably from 12 to 18 carbon atoms.

Mention will be made more particularly of the copolymers consisting of:

(a) from 40 mol % to 99 mol % of 2-acrylamido-2methylpropanesulphonic (AMPS) acid units of formula (IV) below:

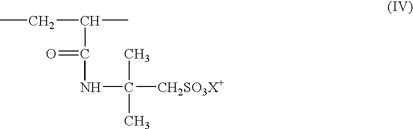

in which X⁺ is a proton, an alkali metal cation, an alkaline-earth metal cation or an ammonium ion; and

(b) from 1 to 60 mol % of units of formula (V) below:

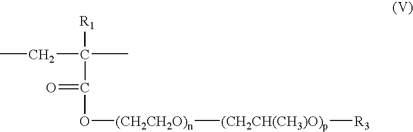

in which n and p, independently of each other, denote a number of moles and range from 0 to 30, preferably from 1 to 25 and more preferably from 3 to 20, with the proviso that n+p is less than or equal to 30, preferably less than 25 and better still less than 20; R₁ denotes a hydrogen atom or a C₁-C₆ linear or branched alkyl radical (preferably methyl) and R₃ denotes a linear or branched alkyl containing from 6 to 30 carbon atoms, preferably from 6 to 22 carbon atoms and better still from 12 to 18 carbon atoms.

Preferably, the polymers in accordance with the invention are partially or totally neutralized with a mineral base (for example sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

Among the crosslinked or non-crosslinked amphiphilic polymers of AMPS that will be used more particularly are the products sold under the names Aristoflex HMS and E-47/2000W1 by the company Clariant.

A-3-Acrylic terpolymers

The ionic amphiphilic polymers according to the invention may also be chosen from acrylic terpolymers obtained from (a) an α,α-ethylenically unsaturated carboxylic acid, (b) a non-surfactant ethylenically unsaturated monomer different from (a), and (c) a nonionic urethane monomer which is the product of reaction of a monohydric nonionic amphiphilic compound with a monoethylenically unsaturated isocyanate. The acrylic terpolymer defined above may be obtained by aqueous dispersion copolymerization of the components (a), (b) and (c), this copolymerization being entirely common and described especially in document EP-A-0 173 109.

Preferably, these amphiphilic polymers are partially or totally neutralized with a mineral base (sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures of these compounds.

As terpolymers that may be used according to the invention, mention may be made of the methacrylic acid/methyl acrylate/behenyl dimethyl-m-isopropenylbenzylisocyanate terpolymer ethoxylated with 400E, i.e. comprising 40 oxyethylene groups, sold by the company Amerchol under the names Viscophobe DB 1000 NP3 and NP4.

Other acrylic terpolymers that may be used in the present invention comprise amphiphilic polymers in which the hydrophilic unit consists of an ethylenic unsaturated anionic monomer, even more preferably a vinylcarboxylic acid and most preferably an acrylic acid, a methacrylic acid or mixtures thereof, and in which the hydrophobic unit is a fatty-chain allyl ether corresponding to the monomer of formula (VI) below:

CH₂═C(R′)CH₂OBₙR (VI)

in which R′ denotes H or CH₃, B denotes an ethyleneoxy radical, n is zero or denotes an integer ranging from 1 to 100, R denotes a hydrocarbon-based radical chosen from alkyl, arylalkyl, aryl, alkylaryl and cycloalkyl radicals, containing from 8 to 30 carbon atoms, preferably 10 to 24 carbon atoms and even more preferably from 12 to 18 carbon atoms. A unit of formula (VI) which is more particularly preferred is a unit in which R′ denotes H, n is equal to 10 and R denotes a stearyl (C₁₈) radical. Anionic amphiphilic polymers of this type are described and prepared, according to an emulsion polymerization process, in patent EP-0 216 479.

Among these anionic amphiphilic polymers that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether of formula (VI), and from 0% to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol allyl ether (10 EO) stearyl alcohol ether (40/50/10), in particular those sold by the company Allied Colloids under the names Salcare SC 80 and Salcare SC 90, which are aqueous 30% emulsions.

B-Water-dispersible ionic amphiphilic polymers

The amphiphilic polymers that may be used according to the invention may as a variant be water-dispersible.

The water-dispersible amphiphilic anionic polymers that may be used according to the invention are, for example, sulphoisophthalic copolyesters consisting essentially of repeating units of isophthalic acid (or of isophthalic esters or isophthalic acid chloride), of diol and of sulphoisophthalic acid.

The diol present in the sulphoisophthalic copolyester may be a cycloaliphatic diol preferably containing from 6 to 20 carbon atoms or an aliphatic diol preferably containing from 3 to 20 carbon atoms. Diols that may be used include ethylene glycol, diethylene glycol, triethylene glycol, 1,4-cyclohexanedimethanol, 1,3-propanediol, 1,4butanediol, 1,5-pentanediol, 1,6-hexanediol, 3-methyl-2,4pentanediol, 2-methyl-1,4-pentanediol, 2,2,4-trimethyl-1,3pentanediol, 2-ethyl-1,3-hexanediol, 2,2-diethyl-1,3propanediol, 1,3-hexanediol, 1,4-bis(hydroxyethoxy)benzene, 2,2-bis(4-hydroxycyclohexyl)propane, 2,4-dihydroxy-1,1,3,3,tetramethylcyclobutane, 2,2-bis(3-hydroxyethoxyphenyl)propane or 2,2-bis(4-hydroxypropoxyphenyl)propane. The isophthalic copolyester may comprise one or more diols as mentioned above and preferably a mixture of ethylene glycol or of 1,4-cyclohexanedimethanol and of diethylene glycol. Advantageously, the sulphoisophthalic copolyester may comprise a diol mixture consisting of at least 45 mol % of diethylene glycol and the remainder to 100 mol % of ethylene glycol or of 1,4-cyclohexanedimethanol.

The sulphoisophthalic acid unit comprises a group SO₃M with M representing a hydrogen atom, an ammonium ion NH₄⁺ or a metal ion, for instance Na⁺, Li⁺ or K⁺, M preferably being Na⁺. The sulphoisophthalic acid that is particularly preferred is the sodium salt of 5-sulphoisophthalic acid.

The sulphoisophthalic acid unit is preferably present in the sulphoisophthalic copolyester in a content that may range from 4 mol % to 25 mol % per 100 mol of acids and 100 mol of diols.

Advantageously, the sulphoisophthalic copolyester may comprise, per 100 mol of acids, from 75 mol % to 90 mol % of isophthalic acid and from 10 mol % to 25 mol % of sulphoisophthalic acid, especially of sodium salt of 5sulphoisophthalic acid, and, per 100 mol of diols, from 45 mol % to 85 mol % of diethylene glycol and from 15 mol % to 55 mol % of 1,4-cyclohexanedimethanol or of ethylene glycol or a mixture thereof.

Such sulphoisophthalic copolyesters are described especially in patents U.S. Pat. No. 3,779,993 and U.S. Pat. No. 5,260,052 and are sold, for example, under the names “Eastman AQ 35S”, “Eastman AQ 38S”, “Eastman AQ 55S”, “Eastman AQ 29D” and “Eastman AQ 38D” by the company Eastman Chemical.

The ionic polymer particles used according to the invention advantageously have a particle size ranging from 10 to 400 nm and better still ranging from 20 to 200 nm, depending on the nature of the ionic polymer. The particles of these polymers may be used as they are or as a dispersion in water. The weight ratio of the polymer particles to the oily phase advantageously ranges from ⅕ to 1/40 and preferably from 1/10 to 1/20. Such a ratio makes it possible to obtain a fluid, fine oil dispersion, i.e. a dispersion containing globules with a particle size of less than 500 nm, which is perfectly stable.

The amount of ionic amphiphilic polymer used in the composition according to the invention is between 0.01% and 20% by weight, preferably between 0.1% and 10% by weight and more preferably between 0.2% and 5% by weight relative to the total weight of the composition.

Since the abovementioned ionic amphiphilic polymer is intended to totally or partially replace the surfactants conventionally used in cosmetic emulsions, according to one preferred embodiment, the amount of surfactant present in the composition according to the invention is less than 1% by weight and preferably less than 0.5% by weight, relative to the total weight of the composition.

The term “surfactant” means any compound or mixture of compounds identified as such in at least one of the following two publications: McCutcheon's: Emulsifiers and Detergents, International Edition 1998, and International Cosmetic Ingredient Dictionary and Handbook (CTFA).

The ionic amphiphilic polymer allows the preparation of emulsions that are stable even in the presence of large amounts of water. Consequently, according to one advantageous embodiment, the composition according to the invention contains at least 45% by weight of water. In addition, the composition according to the invention generally contains at least 1% by weight of fatty phase.

This composition may be in any presentation form conventionally used for topical application, and especially in the form of emulsions of liquid or semi-liquid consistency of the milk type, obtained by dispersing a fatty phase in an aqueous phase (O/W) or conversely (W/O), or suspensions or emulsions of soft, semi-solid or solid consistency of the cream or gel type, or alternatively multiple emulsions (W/O/W or O/W/O emulsions), microemulsions, vesicular dispersions of ionic and/or nonionic type, or wax/aqueous phase dispersions. These compositions are prepared according to the usual methods.

According to one preferred embodiment of the invention, the composition is in the form of an oil-in-water (W/O) emulsion.

As oils which can be used in the composition of the invention, mention may be made for example of:

hydrocarbon-based oils of animal origin, such as perhydrosqualene;

hydrocarbon-based plant-origin oils, such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or shea butter;

synthetic esters and ethers in particular of fatty acids, such as the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, isohexadecane, isododecane, petroleum jelly, polydecenes or hydrogenated polyisobutene such as parleam oil®;

natural or synthetic essential oils such as, for example, eucalyptus oil, hybrid lavender oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;

fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils such as those described in document JP-A-2-295 912;

silicone oils such as volatile or non-volatile polydimethylsiloxanes (PDMSs) containing a linear or cyclic silicone chain, which are liquid or pasty at room temperature, preferably cyclopolydimethylsiloxanes (cyclomethicones) such as cyclohexasiloxane; polydimethylsiloxanes comprising alkyl, alkoxy or phenyl groups, pendant or at the end of a silicone chain, these groups containing from 2 to 24 carbon atoms; phenylsilicones such as phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof.

The term “hydrocarbon-based oil” in the list of abovementioned oils embraces any oil comprising predominantly carbon and hydrogen atoms, and optionally ester, ether, fluoro, carboxylic acid and/or alcohol groups.

Among the oils indicated above, the make-up-removing oils are more particularly fatty acid esters containing at least 12 carbon atoms. These esters are preferably obtained from a straight-chain or branched-chain alcohol containing from 1 to 17 carbon atoms and from a straight-chain or branched-chain fatty acid containing at least 12 carbon atoms and preferably from 14 to 22 carbon atoms. They are preferably mono- or diesters. Examples of make-up-removing oils that may be mentioned include 2-ethylhexyl palmitate (or octyl palmitate), 2-ethylhexyl myristate (or octyl myristate), isopropyl palmitate, isopropyl myristate, diisopropyl adipate, dioctyl adipate, 2-ethylhexyl hexanoate, ethyl laurate, methyl myristate, octyldodecyl octanoate, isodecyl neopentanoate, ethyl myristate, myristyl propionate, 2-ethylhexyl 2-ethylhexanoate, 2-ethylhexyl octanoate, 2-ethylhexyl caprate/caprylate, methyl palmitate, butyl myristate, isobutyl myristate, ethyl palmitate, isohexyl laurate, hexyl laurate and isopropyl isostearate, and mixtures thereof.

The other fatty substances which may be present in the oily phase are, for example, fatty acids containing from 8 to 30 carbon atoms, for instance stearic acid, lauric acid, palmitic acid and oleic acid; waxes, for example lanolin, beeswax, carnauba wax, candelilla wax, paraffin wax, lignite wax or microcrystalline waxes, ceresine or ozokerite, synthetic waxes, for instance polyethylene waxes and FischerTropsch waxes; gums such as silicone gums (dimethiconol); silicone resins such as trifluoromethyl —C₁-₄-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries.

These fatty substances may be chosen in a varied manner by a person skilled in the art in order to prepare a composition having the desired properties, for example consistency or texture properties.

The invention will now be illustrated by means of the non-limiting examples that follow. In these examples, the amounts are indicated as percentages by weight.

### EXAMPLES

### Example 1

### (Comparative): Cosmetic Composition

| | | |
|---|---|---|
| Phase A | | |
| Oxyethylenated (25 OE) oxypropylenated | 1.50 | g |
| (25 OP) dimyristyl tartrate, cetylstearyl | | |
| alcohol, lauryl alcohol and oxyethylenated | | |
| (7 OE) C₁₂₋₁₅ fatty alcohols (Cosmacol PSE | | |
| from Sasol) | | |
| Glyceryl stearate and polyethylene glycol | 2.00 | g |
| stearate (100 OE) | | |
| Stearyl alcohol | 1.00 | g |
| Oils | 10.00 | g |
| Butylparaben | 0.15 | g |
| Ethylhexyl methoxycinnamate | 1.00 | g |
| Phase B | | |
| Preserving agents | 0.50 | g |
| Disodium EDTA | 0.05 | g |
| Crosslinked poly(2-acrylamido-2-methyl- | 0.40 | g |
| propanesulphonic acid) (Hostacerin from | | |
| Hoechst) | | |
| Water | 63.20 | g |
| Phase C | | |
| Xanthan gum | 0.20 | g |
| Phase D | | |
| Mixture of aliphatic polyurethane, N-methyl- | 20.00 | g |
| pyrrolidone and dimethylethanolamine in | | |
| water (Avalure UR410 from Noveon) | | |

The above composition may be prepared in the following manner. The constituents of phase B are heated to about 75° C., except for the crosslinked poly(2-acrylamido-2methylpropanesulphonic acid), which is not introduced until this temperature. After stirring until a homogeneous gel is obtained, phase A heated to about 75° C. is incorporated into phase B. Phase C is then added to this emulsion, with stirring, followed by phase D at 40-45° C. Stirring is continued until the mixture has completely cooled.

### Example 2

### Cosmetic Composition

| | | |
|---|---|---|
| Phase A | | |
| Oxyethylenated, oxypropylenated mixture | 0.79 | g |
| of polyglyceryl isostearyl (4 mol), hexyl | | |
| laurate and polymethylcetyldimethylmethyl- | | |
| siloxane (Abil WE09 from Goldschmidt) | | |
| Cyclopentasiloxane | 7.21 | g |
| Polydimethylsiloxane | 0.90 | g |
| Apricot kernel oil | 4.00 | g |
| Phase B | | |
| Water | 68.15 | g |
| Phenoxyethanol | 1.00 | g |
| Pentasodium salt of ethylenediaminetetra- | 0.05 | g |
| methylenephosphonate | | |
| Acrylic acid/stearyl methacrylate copolymer | 0.60 | g |
| (Pemulen TR1 from Noveon) | | |
| Sodium hydroxide | 0.30 | g |
| Phase C | | |
| Mixture of aliphatic polyurethane, N-methyl- | 17.00 | g |
| pyrrolidone and dimethylethanolamine in water | | |
| (Avalure UR410 from Noveon) | | |

The above composition may be prepared in the following manner. The constituents of phase B are heated to about 75° C., except for the acrylic copolymer, which is not introduced until this temperature. After stirring until a homogeneous gel is obtained, phase A heated to about 75° C. is incorporated into phase B. Phase C is then added to this emulsion, with stirring, at 40-45° C. Stirring is continued until the mixture has completely cooled.

### Example 3

### Cosmetic Composition

Multiple Emulsion:

| | | |
|---|---|---|
| Primary emulsion (A) | 22.50 | g |
| Cyclopentasiloxane | 3.50 | g |
| Apricot kernel oil | 4.00 | g |
| Water | 51.05 | g |
| Phenoxyethanol | 1.00 | g |
| Pentasodium salt of ethylenediaminetetra- | 0.05 | g |
| methylenephosphonate | | |
| Acrylic acid/stearyl methacrylate copolymer | 0.60 | g |
| (Pemulen TR1 from Noveon) | | |
| Sodium hydroxide | 0.30 | g |
| Mixture of aliphatic polyurethane, N-methyl- | 17.00 | g |
| pyrrolidone and dimethylethanolamine in | | |
| water (Avalure UR410 from Noveon) | | |

Primary Emulsion (A):

| | | |
|---|---|---|
| Water | 75.20 | g |
| Oxyethylenated, oxypropylenated mixture | 3.50 | g |
| of polyglyceryl isostearyl (4 mol), hexyl | | |
| laurate and polymethylcetyldimethylmethyl- | | |
| siloxane (Abil WE09 from Goldschmidt) | | |
| Cyclopentasiloxane | 16.50 | g |
| Polydimethylsiloxane | 4.00 | g |
| Magnesium sulphate | 0.80 | g |

Procedure

Preparation of the Primary Emulsion:

The Abil WE09, the cyclopentasiloxane and the polydimethylsiloxane are homogenized at room temperature and with stirring. The water and the magnesium sulphate are incorporated slowly, with vigorous stirring.

Preparation of the Multiple Emulsion:

The acrylic copolymer, the phenoxyethanol and the phosphonic sequestering agent are dispersed at room temperature and with stirring. The mixture is left to swell for about 45 minutes with stirring, and the Avalure UR410 is then added. The resulting mixture is then neutralized with the sodium hydroxide, and the primary emulsion is then diluted with the cyclopentasiloxane and the apricot kernel oil. This mixture is then incorporated slowly into the aqueous phase with stirring.

### Example 4

### Cosmetic Composition

| Phase A | | |
|---|---|---|
| Water | 35.00 | g |
| Sulphopolyester Eastman AQ55S from | 2.00 | g |
| Eastman Chemical | | |
| Preserving agents | 1.00 | g |
| Phase B | | |
| Cyclopentasiloxane | 20.00 | g |
| Phase C | | |
| Xanthan gum | 0.50 | g |
| Water | 21.50 | g |

Phase D

Mixture of aliphatic polyurethane, N-methylpyrrolidone and dimethylethanolamine in water (Avalure UR410 from Noveon) 20.00 g

The above composition may be prepared in the following manner. The constituents of phase A are mixed together and the mixture is heated at 70° C. with magnetic stirring until the polymer is completely dispersed, and the solution is then cooled to room temperature. Phases B and C are prepared separately (under hot conditions). Phase A is then introduced into phase B with vigorous stirring, followed by phase C. The emulsion is homogenized under a pressure of 600 bar (2 to 4 homogenizations), returning the emulsion to room temperature between each homogenization. Phase D is then added to the emulsion.

### Example 5

### Demonstration of the Tensioning Effect

a) Protocol

The tensioning power of the cosmetic compositions of Examples 1 to 4 may be measured using an extensometer (MTT 610 supplied by the company Dia-Stron). The principle of the method consists in measuring the length of a sample of stratum corneum isolated from human skin obtained from a surgical operation, before and after treatment with the test compositions.

To do this, the sample is placed between the two jaws of the machine, one of which is fixed and the other mobile, under an atmosphere at 30° C. and 40% relative humidity. A tensile force is exerted on the sample, and the curve of the force (in grams) as a function of the length (in millimetres) is recorded, the zero length corresponding to the contact between the two jaws of the machine. The tangent to the curve in its linear region is then plotted. The intersection of this tangent with the X-axis corresponds to the apparent length L₀ of the sample with zero force. The sample is then relaxed, after which 2 mg/cm² of the test composition are applied to the stratum corneum. After drying for 15 minutes, the above steps are repeated to determine the length L₁ of the sample after treatment.

The percentage of retraction is defined by:

% retraction=100×(L₁−L₀)/L_{0.}

To characterize a tensioning effect, this percentage must be negative and the tensioning effect is proportionately greater the larger the absolute value of the percentage of retraction. The tensioning power of the compositions described in the present patent application is characterized by a retraction of isolated stratum corneum of more than 1% as an absolute value at 30° C. under a relative humidity of 40% (see Table 1).

b) Results

The results obtained are collated in Table 1 below.

TABLE 1
Retraction of isolated stratum corneum

| | % of retraction of a sample |
|---|---|
| Compositions | of isolated stratum corneum |
| Example 1: O/W direct | 0.57% ± 0.26 |
| emulsion containing 7% | |
| Avalure UR410 (surfactant | |
| content: 3.5%) | |
| Example 2: O/W direct | 1.56% ± 0.31 |
| emulsion containing 6% | |
| Avalure UR410 (surfactant | |
| content: 0.8%) | |
| Example 3: W/O/W multiple | 1.50% ± 0.35 |
| emulsion containing 6% | |
| Avalure UR410 (surfactant | |
| content: 0.8%) | |
| Example 4: O/W emulsion | 2.76% ± 0.77 |
| containing 7% Avalure UR410 | |
| (no surfactant) | |

As is seen from this table, the tensioning effect of a surfactant-rich O/W emulsion containing 7% Avalure UR 410 is not significant (Comparative Example 1). In contrast, the O/W and W/O/W emulsions according to the invention (Examples 2 to 4) containing 6% or 7% Avalure UR410 and less than 1% surfactant produce a tensioning effect, making it possible to envisage their use for restoring the tautness of the skin and smoothing out wrinkles and fine lines.

### Example 6

### Sensory Evaluation

The composition of Example 3 is tested on a panel of 40 to 60 year old women with wrinkles and fine lines around the eyes. A smoothing effect of the fine lines under the eyes is observed.

### Example 7

### Stability Test

The stability over time and the temperature stability of the compositions of Examples 1 to 4 above are evaluated: no physicochemical change in these compositions is observed over two months at 4° C., 25° C., 37° C. and 45° C., in the sense that their pH, their viscosity (measured using a Rheomat RM180 rheometer from the company Mettler), their appearance and their odour remained unchanged.

Unless stated to the contrary, any use of the words such as “including,” “containing,” comprising,” “having” and the like, means “including without limitation” and shall not be construed to limit any general statement that it follows to the specific or similar items or matters immediately following it. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A composition that is suitable for topical application to skin, comprising an aqueous phase, a fatty phase, a dispersion of tensioning polymer particles, and at least one ionic amphiphilic polymer.

2. The composition of claim 1, wherein said dispersion of said tensioning polymer particles comprises at least one polymer selected from polyurethane polymers or copolymers, acrylic polymers or copolymers, sulphonated isophthalic acid polymers or grafted silicone polymers.

3. The composition of claim 2, wherein said dispersion of said tensioning polymer particles comprises at least one dispersion of polyester-polyurethane particles.

4. The composition of claim 2, wherein said grafted silicone polymer is a polydimethylsiloxane onto which are grafted, via a linking chain of thiopropylene type, mixed polymer units of poly(meth)acrylic acid type and polyalkyl (meth)acrylate type.

5. The composition of claim 4, wherein said grafted silicone polymer is a copolymer of polydimethylsiloxane comprising propylthio groups, methyl acrylate, methyl methacrylate and methacrylic acid.

6. The composition of claim 2, wherein said synthetic polymer is selected from interpenetrated polymer networks.

7. The composition of to claim 6, wherein said interpenetrated polymer is an aqueous dispersion of particles based on polyurethane and polyacrylic, with a weight-average size of between 90 and 110 nm, a number-average size of about 80 nm and a glass transition temperature, Tg, ranging from about −60° C. to +100° C.

8. The composition of claim 1, wherein said amphiphilic polymer is selected from acrylic copolymers, hydrophobic AMPS derivatives, acrylic terpolymers or sulphoisophthalic copolyesters.

9. The composition of claim 8, wherein said acrylic copolymers are obtained by copolymerization of a monomer (a) selected from α,β-ethylenically unsaturated carboxylic acids with a monomer or (b) derived from the reaction of an ethylenically unsaturated carboxylic acid monomer with an optionally alkoxylated, preferably polyethoxylated aliphatic fatty alcohol, wherein the carbon-based chain of said aliphatic fatty alcohol contains at least 6 carbon atoms.

10. The composition of claim 9, wherein said acrylic copolymers consist of polymers formed from a mixture of monomers comprising acrylic acid and an ester of formula (III): 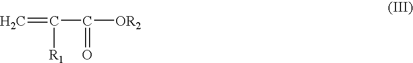
in which R₁ denotes H or CH₃, R₂ denoting an alkyl radical containing from 12 to 22 carbon atoms, and a crosslinking agent.

11. The composition of claim 10, wherein said acrylic copolymers consist of 95% to 60% by weight of acrylic acid, 4% to 40% by weight of C₁₀-C₃₀ alkyl acrylate and 0% to 6% by weight of crosslinking polymerizable monomer.

12. The composition of claim 11, wherein said acrylic copolymers consist of 98% to 96% by weight of acrylic acid, 1% to 4% by weight of C₁₀-C₃₀ alkyl acrylate and 0.1% to 0.6% by weight of crosslinking polymerizable monomer.

13. The composition of claim 11, wherein said crosslinking polymerizable monomer is selected from polyallylsucrose or polyallylpentaerythritol.

14. The composition of claim 9, wherein said acrylic copolymer is obtained by copolymerization of a monomer (a) selected from α,β-ethylenically unsaturated carboxylic acids with a monomer or (b) derived from the reaction of itaconic acid with an alkoxylated, preferably polyethoxylated aliphatic fatty alcohol comprising from 1 to 50 alkylene oxide units, wherein the carbon-based chain of said aliphatic fatty alcohol contains at least 6 carbon atoms.

15. The composition of claim 8, wherein said hydrophobic AMPS derivative is selected from crosslinked or non-crosslinked amphiphilic polymers of 2-acrylamido-2methylpropanesulphonic (AMPS) acid or of at least one ethylenically unsaturated monomer comprising at least one hydrophobic portion containing from 6 to 30 carbon atoms.

16. The composition of claim 15, wherein said hydrophobic AMPS derivative is a copolymer consisting of:
(a) from 40 mol % to 99 mol % of 2-acrylamido-2-methylpropanesulphonic (AMPS) acid units of formula (IV): 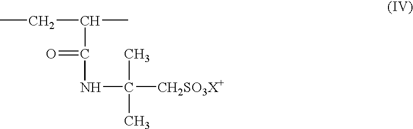
in which X⁺ is a proton, an alkali metal cation, an alkalineearth metal cation or an ammonium ion; and
(b) from 1 mol % to 60 mol % of units of formula (V): 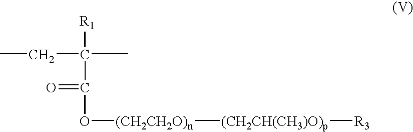
in which n and p, independently of each other, denote a number of moles and range from 0 to 30, with the proviso that n+p is less than or equal to 30; R₁ denotes a hydrogen atom or a C₁-C₆ linear or branched alkyl radical and R₃ denotes a linear or branched alkyl containing from 6 to 30 carbon atoms.

17. The composition of claim 8, wherein said acrylic terpolymer is obtained from (a) an (α,α-ethylenically unsaturated carboxylic acid, (b) a non-surfactant ethylenically unsaturated monomer different from (a) or (c) a nonionic urethane monomer which is the product of reaction of a monohydric nonionic amphiphilic compound with a monoethylenically unsaturated isocyanate.

18. The composition of claim 17, wherein said acrylic terpolymer is a methacrylic acid/methyl acrylate/behenyl alcohol dimethyl-m-isopropenylbenzylisocyanate terpolymer ethoxylated with 40 EO.

19. The composition of claim 8, wherein said acrylic terpolymer is formed from (a) 20% to 60% by weight of acrylic acid and/or of methacrylic acid, (b) 5% to 60% by weight of lower alkyl (meth)acrylates, (c) 2% to 50% by weight of fatty-chain allyl ether of formula (VI)
CH₂═C(R′)CH₂OBₙR (VI)
in which R′ denotes H or CH₃, B denotes an ethyleneoxy radical, n is zero or denotes an integer ranging from 1 to 100, R denotes a hydrocarbon-based radical chosen from alkyl, arylalkyl, aryl, alkylaryl and cycloalkyl radicals, containing from 8 to 30 carbon atoms, and (d) 0% to 1% by weight of a crosslinking agent which is a copolymerizable polyethylenically unsaturated monomer chosen from diallyl phthalate, allyl (meth)acrylate, divinylbenzene, (poly)ethylene glycol dimethacrylate and methylenebisacrylamide.

20. The composition of claim 19, wherein said acrylic terpolymer is a crosslinked terpolymer of methacrylic acid, ethyl acrylate, and stearyl alcohol ether of polyethylene glycol allyl ether (10 EO)(40/50/10).

21. The composition of claim 8, wherein said sulphoisophthalic copolyester consists essentially of repeated units of isophthalic acid or isophthalic acid esters or isophthalic acid chloride, diol, and sulphoisophthalic acid.

22. The composition of claim 21, wherein said sulphoisophthalic copolyester comprises, per 100 mol of acids, from 75 mol % to 90 mol % of isophthalic acid and from 10 mol % to 25 mol % of sulphoisophthalic acid or sodium salt of 5sulphoisophthalic acid, and, per 100 mol of diols, from 45 mol % to 85 mol % of diethylene glycol and from 15 mol % to 55 mol % of 1,4-cyclohexanedimethanol or ethylene glycol, or a mixture thereof.

23. The composition of claim 1, wherein said composition contains from 0.5% to 2% by weight of ionic amphiphilic polymer relative to the total weight of the composition.

24. The composition of claim 1, further comprising less than 1% by weight of surfactant relative to the total weight of the composition.

25. The composition of claim 24, wherein said surfactant is present at less than 0.5% by weight relative to the total weight of the composition.

26. The composition of claim 1, wherein said composition contains at least 45% by weight of water relative to the total weight of the composition.

27. The composition of claim 1, wherein said fatty phase is present at an amount of at least 1% by weight relative to the total weight of the composition.

28. The composition of claim 1, wherein said tensioning polymer particle is present at a concentration from 0.1% to 50% by weight relative to the total weight of the composition.

29. The composition the claim 28, wherein said concentration is from 1% to 20% by weight relative to the total weight of the composition.

30. The composition of claim 1, wherein said ionic amphiphilic polymer is present at an amount between 0.01% and 20% by weight relative to the total weight of the composition.

31. The composition of claim 30, wherein said amount is between 0.1% and 10% by weight relative to the total weight of the composition.

32. The composition of claim 31, wherein said amount is between 0.2% and 5% by weight relative to the total weight of the composition.

33. A composition that is suitable for topical application to skin comprising:
a) an aqueous phase,
b) a fatty phase
c) a dispersion of tensioning polymer particles, said tensioning polymer particles exhibit a retraction of isolated stratum corneum of 1.5% when tested at 30° C. at a relative humidity of 40%, and at a concentration of 7% in water, and
d) at least one amphiphilic polymer.

34. The composition of claim 33, wherein said tensioning polymer particles are present at a concentration from 0.1% to 50% by weight relative to the total weight of the composition.

35. The composition of claim 34, wherein said concentration is from 1% to 20% by weight relative to the total weight of the composition.

36. The composition of claim 33, optionally including a surfactant in an amount of less than 1% by weight relative to the total weight of the composition.

37. The composition of claim 36, wherein said amount is less than 0.5% by weight relative to the total weight of the composition.

38. The composition of claim 33, wherein said composition contains at least 45% by weight of water relative to the total weight of the composition.

39. The composition of claim 33, wherein said fatty phase is present in an amount of at least 1% relative to the total weight of the composition is present.

40. The composition of claim 33, wherein said ionic amphiphilic polymer is present at an amount between 0.01% and 20% by weight relative to the total weight of the composition.

41. The composition of claim 40, wherein said amount is between 0.1% and 10% by weight relative to the total weight of the composition.

42. The composition of claim 41, wherein said amount is between 0.2% and 5% by weight relative to the total weight of the composition.

43. A method for treating wrinkled skin comprising applying said composition of claim 1 to said skin to smooth out wrinkles and fine lines.

44. A method for restoring skin tautness comprising applying said composition of claim 1 to said skin.

45. A method for treating wrinkled skin comprising applying said composition of claim 24 to said skin to smooth out wrinkles and fine lines.

46. A method for restoring skin tautness comprising applying said composition of claim 24 to said skin.
